(19) United States
(12) Patent Application Publication
(10) Pub. No.: US 2024/0404642 A1
(43) Pub. Date: Dec. 5, 2024

(54) GENOME GRAPH ANALYSIS METHOD, DEVICE AND MEDIUM BASED ON IN-MEMORY COMPUTING

(71) Applicant: ZHEJIANG LAB, Hangzhou (CN)

(72) Inventors: Long ZHENG, Hangzhou (CN); Yu HUANG, Hangzhou (CN); Wei ZHOU, Hangzhou (CN)

(21) Appl. No.: 18/460,671

(22) Filed: Sep. 4, 2023

(30) Foreign Application Priority Data
May 30, 2023 (CN) 202310623475.5

(51) Int. Cl. G16B 50/30

(52) U.S. Cl. CPC G16B 50/30

(57) ABSTRACT

A method, a device and a medium for genome graph analysis based on in-memory computing. The method comprises the following steps: firstly, combining a linear reference genome with genetic variation to construct a genome graph; then, generating indexes for a plurality of vertices of the genome graph, and constructing an index table according to the generated indexes; then dividing the read length into a plurality of substrings with the length of k-mer, and querying the index table to obtain a seed position, generating a reference subgraph according to the seed position, and identifying a candidate mapping position according to the reference subgraph to filter a candidate mapping area; finally, using a PUM mode to run approximate string matching between the read length and all unfiltered candidate mapping positions, so as to complete the optimal alignment of a reference gene sequence and a query gene sequence.

### CROSS-REFERENCE TO RELATED APPLICATIONS

The present application claims priority to Chinese Patent Application No. 202310623475.5, filed on May 30, 2023, the content of which is incorporated herein by reference in its entirety.

### TECHNICAL FIELD

The present disclosure relates to the field of high-performance computing technology and bioinformatics, in particular to a genome graph analysis method, device and medium based on in-memory computing.

### BACKGROUND

Genome graph analysis refers to the mapping of a DNA sequence (called a read length) to a reference genome, which is crucial in medical care and biological sciences. The current reference genomes are usually expressed as linear DNA sequences. However, genetic variation and diversity occur in populations, such as single nucleotide polymorphism, insertion and deletion, and structural variation. A single reference genome cannot represent multiple groups of individuals, allowing the drawing process biased. The genome graph combines the reference genome with genetic variation as a graph-based data structure, in which a vertex represents one or more base pairs, such as A, G, T, C, and an edge connects the vertices to represent the base pair sequence. Multiple output edges in one vertex represent different genetic variations, thus it allows mapping with different sequences in individuals instead of a single reference genome.

In the analysis of a genome graph, the seed and extension technology is usually adopted, which includes five steps: construction, indexing, seeding, filtering and comparison. It is worth noting that the graph and index thereof are constant data structures, thus the construction and index are only preprocessed once. Therefore, this preprocessing overhead can be shared by multiple mapping executions. Therefore, it only needs to focus on accelerating two key steps, seeding and comparison, in genome graph analysis in this work.

In order to determine the key bottleneck of genome graph analysis, the software variant of the most advanced solution SerGraM is implemented on CPU for feature analysis. SerGraM consists of two main components. SerGraM uses the method based on minimizers to seed the candidate mapping positions (denoted as MinSeed), and uses a Bitap algorithm to align the read length and candidate mapping positions (denoted as BitAlig). After experimental analysis, MinSeed contains sparse search for large index tables. The candidate entries that need to be accessed may be distributed throughout the index table due to the poor predictability of the input read length. Index tables are usually in the order of tens of GBs, making prefetching and caching technology infeasible. In contrast, BitAlig involves a large number of simple bitwise alignment operations on a narrow data area. The comparison is mainly between the read length and the candidate region, so it only accesses the subgraph data, not the whole genome data. Therefore, the MinSeed seeding process is located in the memory binding area, while the BitAlig alignment process is located in the computational binding area. In particular, MinSeed shows relatively fixed arithmetic strength as it queries the seed position in the index by performing element-by-element comparison. MinSeed operation involves a large number of irregular index accesses due to the inherent irregularity of input reading. On the other hand, BitAlig often uses regular memory access to perform bitwise operations. The operation intensity increases with the increase of the read length as the dimension of the bit vector involved in bit-by-bitwise operation increases. Therefore, the two key components of genome graph analysis show different characteristics. The seeding process involves a large number of irregular memory accesses to large data areas, which is constrained by the access delay of a long off-chip memory. Alignment process combines a highly parallel bitwise operation with a conventional memory access mode, which has insufficient computing power.

PIM is an attractive candidate to reduce the overhead of data movement and provide high throughput computing power. On the one hand, the computing unit in PIM (PNM) integrated near or inside the memory has the characteristics of low latency and high bandwidth memory access and the potential to reduce the memory access delay, so as to be used to reduce the data transmission overhead in the seeding step. In addition, the seeding process typically has low operational intensity and simple search operation, allowing the computational logic to be integrated into the memory in a cost-effective manner. On the other hand, Process-In-Memory PIM (Process-Using-Memory PUM) processes the data in the memory subarray without reading the data, which provides in-situ computing capability with large-scale parallelism is thus utilized through the alignment step. In addition, the operations of the alignment process are mainly composed of bitwise operations, allowing efficient deployment in memory lines.

As mentioned above, PIM provides an attractive candidate for the acceleration of genome graph analysis. The disclosed methods have not studied how to design a PIM-supporting architecture sufficiently to allow the seeding and comparison processes to be accelerated effectively at the same time. Seeding step is more suitable for low access delay brought by PNM, while alignment is more suitable for high throughput processing brought by the PUM method. In order to have the best of both worlds, the present disclosure aims to propose a memory accelerator based on genome graph analysis slightly modifying the commercial DIMM architecture, and the memory accelerator puts the processing unit in each DIMM level to query the seed and at the same time uses DRAM timing conflict to perform bitwise alignment operation.

In addition, on the one hand, there are differences in understanding of those skilled in the art. On the other hand, since the applicant has studied a large number of documents and patents when making the present disclosure, but not all the details and contents are listed in detail due to the limitation of space. However, this is by no means that the present disclosure does not have the characteristics of the prior art. On the contrary, the present disclosure already has all the characteristics of the prior art, and the applicant reserves the right to add the related art to the background technology.

### SUMMARY

In view of the shortcomings of the prior art, the present disclosure aims to provide a genome graph analysis method, a device and a medium based on in-memory computing.

The object of the present disclosure is achieved through the following technical solution: a first aspect of the embodiment of the present disclosure provides a genome graph analysis method based on in-memory computing; the method includes the following steps:
(1) Construction: combining a linear reference genome with genetic variation to construct a genome graph.
(2) Indexing: generating an index for a plurality of vertices of the genome graph, and constructing an index table according to the generated indexes.
(3) Seeding: dividing a read length into a plurality of substrings with a length of k-mer, querying the index table to obtain a seed position, generating a reference subgraph according to the seed position, and identifying a candidate mapping position according to the reference subgraph to filter a candidate mapping area.
(4) Alignment: running approximate string matching between the read length and all unfiltered candidate mapping positions adopting a PUM mode, so as to achieve an optimal alignment of a reference gene sequence and a query gene sequence.

Further, the step (1) includes the following sub-steps:
(1.1) acquiring basic information of the linear reference genome using a genome browser software, and the basic information comprises a genome sequence and gene annotation information.
(1.2) Sequencing and analyzing the basic information to acquire genetic variation information.
(1.3) Combining the genetic variation information with the genome sequence of the linear reference genome by adopting a comparison-based method and an assembly-based method to acquire individual genome sequences and variation information in the individual genome sequences.
(1.4) Annotating genome on the variation information in the individual genome sequences by a gene annotation tool to acquire annotation information.
(1.5) Combining the linear reference genome, the individual genome sequences and the annotation information to construct a genome graph.

Further, the step (2) includes the following sub-steps:
(2.1) Calculating, for each genome graph, hash values of all vertices in the genome graph and mapping the hash values into one bucket.
(2.2) Sorting, for each bucket, all the vertices in the bucket according to the hash values, and assigning one index to each vertex.
(2.3) Adding, for each vertex, an index of each vertex in the each genome graph to a corresponding hash table entry.
(2.4) Sorting all hash table entries according to vertex identifiers and storing the hash table entries in the constructed index table.

Further, the step (3) includes the following sub-steps:
(3.1) Receiving, by the read length traverser, an input read length from a host, a seed processing unit includes a read length traverser and a seed finder.
(3.2) Traversing, by the read length traverser, the input read length in order to obtain a plurality of minimizers, and scoring the plurality of minimizers according to a scoring mechanism to obtain a plurality of optimized minimizers.
(3.3) Storing the acquired minimizers in a minimizer cache region and counting the minimizers.
(3.4) Filtering, by the read length traverser, minimizers smaller than a first preset threshold and minimizes larger than a second preset threshold after traversing all characters in the read length, to acquire filtered minimizers.
(3.5) Reading, by the seed finder, the filtered minimizers output by the read length traverser to obtain unfiltered minimizers.
(3.6) Querying, by the seed finder, a seed position of the unfiltered minimizers and the reference sequence information of the seed position of the unfiltered minimizers from the index table.
(3.7) Generating, by the seed finder, the reference subgraph according to the seed position of the unfiltered minimizes and the reference sequence information of the seed position of the unfiltered minimizers acquired in the sub-step (3.6), and identifying the candidate mapping position according to the reference subgraph to filter the candidate mapping area.

Further, the minimizers are a set of sequences in the substrings with the length of k-mer, and the minimizers satisfy the following conditions: each of the minimizers must appears at least twice in the substrings with the length of k-mer; each of the minimizers cannot be a subsequence of other minimizers; and a sum of lengths of all the minimizers must be less than or equal to a total length of the sequences in the substrings with the length of k-mer.

Further, the step (4) includes the following sub-steps:
(4.1) Flowing an alignment instruction into an instruction cache area of a register clock driver by adopting the PUM mode.
(4.2) Decoding the alignment instruction by adopting a PUM instruction decoder added in the register clock driver to acquire a subarray corresponding to the alignment instruction, and loading data into the subarray.
(4.3) Generating four pattern bit masks of A, G, T and C for querying the read length based on a bitmap method.
(4.4) Iterating each vertex of the reference subgraph, and calculating the insertion, deletion, replacement and matching bit vectors of the each vertex by a bitwise operation, so as to achieve the optimal alignment of the reference gene sequence and the query gene sequence.

Further, the step (4.3) specifically includes: firstly, preprocessing, including generating one pattern bit mask for each character of a pattern string; and then calculating an editing distance, updating and saving a state bit vector of the partial matching information of text characters checked so far at each text iteration using the preprocessed pattern bit mask, and checking each of the text characters one by one by the bitwise operation.

Further, the step (4.4) specifically includes: iterating the each vertex of the reference subgraph, and dividing the subarray into three groups: a data group, a control group and a bitwise group, and the data group corresponds to a row for storing conventional data, the control group contains four pattern bit mask vectors and two pre-initialized rows to control a bitwise alignment operation, and the bitwise group consists of rows that perform the bitwise operation in a row parallel manner; calculating the insertion, deletion, replacement and matching bit vectors of the each vertex by the data group, the control group and the bitwise group as well as the bitwise operation, so as to achieve the optimal alignment of the reference gene sequence and the query gene sequence.

A second aspect of the embodiment of the present disclosure provides a genome graph analysis device based on in-memory computing, including one or more processors for implementing the above genome graph analysis method based on in-memory computing.

A third aspect of the embodiment of the present disclosure provides a computer-readable storage medium on which a program is stored, and the program, when executed by a processor, is configured to implement the above genome graph analysis method based on in-memory computing.

The present disclosure has the advantages that the present disclosure innovatively integrates processing-near-memory and in-situ computing, and carries out low-cost modification; according to the present disclosure, a customized seed processing unit is placed beside each DRAM level to explore the parallelism of the levels, so that the low access delay can reduce the irregular memory access overhead in the seeding step; the present disclosure takes the index as the center, which effectively reduces the transmission of index data between rows and columns; according to the present disclosure, the row access command sequence of the modified subarray structure is specialized, and the row parallel in-situ computing ability is allowed to adjust the bitwise operation in the alignment step; the present disclosure further introduces the distance sensing technology to eliminate the complex data dependence in the genome graph; the present disclosure extends the instruction set to support customized memory operation; and the present disclosure can accelerate the analysis of the genome graph.

### BRIEF DESCRIPTION OF DRAWINGS

FIG. 1 is a schematic structural diagram of a genome graph analysis method based on in-memory computing according to a preferred embodiment of the present disclosure;

FIG. 2 is an architecture diagram of a genome graph analysis method based on in-memory computing in a preferred embodiment of the present disclosure; and

FIG. 3 is a schematic structural diagram of the genome graph analysis device based on in-memory computing of the present disclosure.

### DESCRIPTION OF EMBODIMENTS

Reference will now be made in detail to exemplary embodiments, examples of which are illustrated in the accompanying drawings. When the following description refers to the drawings, unless otherwise indicated, the same numbers in different drawings indicate the same or similar elements. The embodiments described in the following exemplary embodiments do not represent all embodiments consistent with this application. Rather, they are merely examples of devices and methods consistent with some aspects of the present disclosure as detailed in the appended claims.

The terminology used in this application is for the purpose of describing specific embodiments only and is not intended to limit this application. The singular forms “a”, “said” and “the” used in this application and the appended claims are also intended to include the plural forms, unless the context clearly indicates other meaning. It should also be understood that the term “and/or” as used herein refers to and includes any or all possible combinations of one or more associated listed items.

It should be understood that although the terms first, second, third, etc. may be used in this application to describe various information, this information should not be limited to these terms. These terms are only used to distinguish the same type of information from each other. For example, without departing from the scope of this application, the first piece of information can also be called the second piece of information, and similarly, the second piece of information can also be called the first piece of information. Depending on the context, the word “if” as used herein can be interpreted as “when” or “in case of” or “in response to a determination”.

The present disclosure will be described in detail with reference to the attached drawings. In the case of no conflict, the features in the following embodiments and implementations can be combined with each other.

Genome graph analysis refers to mapping a DNA sequence (called A read length) to a reference genome. Aiming at the problem that the current reference genome is usually expressed as a linear DNA sequence, and a single reference genome cannot represent multiple groups of individuals, which may lead to deviation in the drawing process, the present disclosure aims to combine the reference genome with genetic variation by using a graph-based data structure, so as to allow mapping with different sequences in individuals instead of a single reference genome.

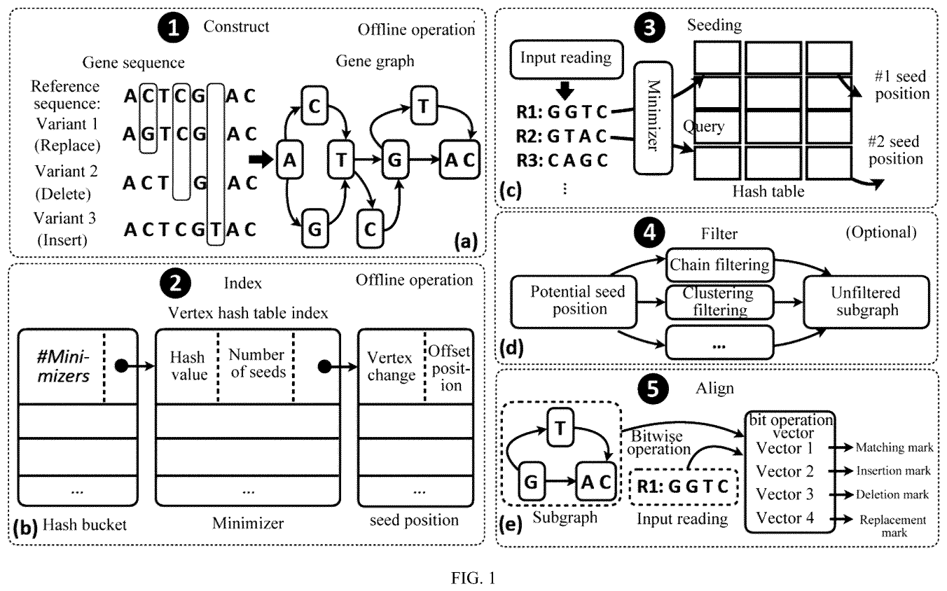

Referring to FIG. 1, the genome graph analysis method based on in-memory computing of the present disclosure specifically includes the following steps:
(1) Construction: the linear reference genome is combed with genetic variation to construct a genome graph.
(1.1) The basic information of the linear reference genome is acquired using a genome browser software, and the basic information includes genome sequences, gene annotation information and other important genome features.

In this embodiment, the genome browser software can adopt UCSC Genome Browser software. It should be understood that other genome browser software that can obtain the basic information of the linear reference genome can also be used.
(1.2) The basic information is sequenced and analyzed to acquire genetic variation information.

In this embodiment, various sequencing techniques can be used for sequencing, such as whole genome sequencing, RNA sequencing, methylation sequencing, etc. Various analysis tools, such as GATK, Samtools, Picard and the like can be used for analysis. Finally, the genetic variation information includes SNP, INDEL, structural variation and the like.
(1.3) The genetic variation information is combined with the genome sequences of the linear reference genome by the comparison-based method and the assembly-based method, so as to acquire individual genome sequences and variation information in the individual genome sequences.

In this embodiment, the comparison-based method is to compare the sequencing data with the linear reference genome to determine the location and type of variation. The assembly-based method is to assemble the sequencing data to obtain more complete individual genome sequences, and then compare it with the linear reference genome to determine the location and type of variation. It should be noted that both alignment-based method and assembly-based method need to combine genetic variation information with the genome sequences to obtain variation information in individual genome sequences.
(1.4) A gene annotation tool is used to annotate the variation information in individual genome sequences to obtain annotation information. In this way, the effects on genes and other functional regions can be determined.

Further, the gene annotation tool includes NCBI's gene annotation tools, Ensembl, Maker, and so on.
(1.5) The linear reference genome, individual genome sequences and annotation information are combined to construct the genome graph.

It should be understood that various mapping software can be used to construct the genome graph, such as Circos, ggplot2, etc.
(2) Indexing: indexes are generated for a plurality of vertices of the genome graph, and an index table is constructed according to the generated indexes.

It should be understood that the index table is a data structure, which is used to store the seed position and the corresponding reference sequence information of the substring sequence with the length of k-mer, so that the needed graph can be found quickly in the following steps.

In this embodiment, the hash table is mainly used to construct the index table, which specifically includes the following steps:
(2.1) For each genome graph, the hash values of all vertices in the genome graph is calculated and the hash values into one bucket is mapped.
(2.2) For each bucket, all the vertices in the bucket is sorted according to the hash values, and one index is assigned to each vertex.
(2.3) For each vertex, the index thereof in the each genome graph is added to the corresponding hash table entry.

It should be understood that each hash table entry contains the identifier of a vertex and the index of the vertex in each graph.
(2.4) all hash table entries is sorted according to vertex identifiers and the hash table entries in the constructed index table is sorted.

Using the above method, the same vertex in multiple genome graphs can be quickly found out, thus constructing the comparison relationship between them. In addition, there are some optimization techniques, such as Bloom filter and compressed hash table, to reduce the size and memory occupation of index table, thus improving the speed and efficiency of comparison.
(3) Seeding: the read length is divided into a plurality of substrings with the length of k-mer, and the index table is queried to obtain the seed position, and the reference subgraph is generated according to the seed position, and the candidate mapping positions are identified according to the reference subgraph, so as to filter the candidate mapping area, which is beneficial to reducing the number of alignments required in the following steps.

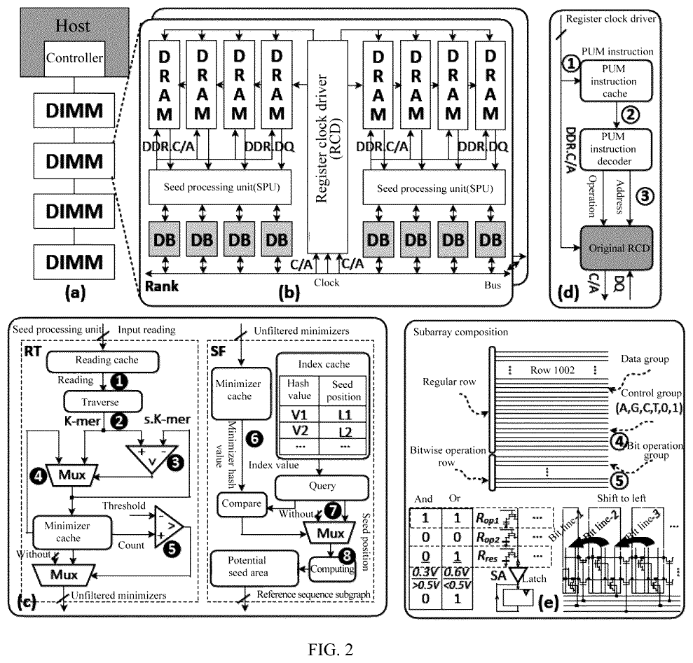

It should be understood that methods such as sliding window methods and hash function methods can be used to divide the read length into multiple substrings with the length of k-mer, which is convenient for subsequent queries in the index table.
(3.1) A seed processing unit (SPU) mainly includes a read length traverser (RT) and a seed finder (SF), and the read length traverser receives the input read length from a host, as shown in FIG. 2.

It should be noted that the seed processing unit (SPU) is embedded in the cache chip of DIMM, and is used to perform the seed index query operation. One of the main modules of the seed processing unit is the read length traverser (RT), which receives the input read length from the host.
(3.2) The read length traverser traverses the input read length in order to obtain a plurality of minimizers, and scores the plurality of minimizers according to the scoring mechanism to obtain a plurality of optimized minimizers.

In this embodiment, the minimizer is a set of sequences in the substring with the length of k-mer, which have certain commonness and similarity and can be used to represent the whole substring sequence. In an embodiment, the minimizers must meet the following conditions: each minimizer must appear at least twice in a substring with the length of k-mer; each minimizer cannot be a subsequence of other minimizers; and the sum of the lengths of all minimizers must be less than or equal to the total length of the substring sequence with the length of k-mer.

In this embodiment, after acquiring the minimizer, the scoring mechanism is used to score the minimizer to determine the quality and reliability of a plurality of minimizers. In an embodiment, the scoring mechanism specifically includes the following aspects:
(a) De-duplication: using a hash function and a hash table, de-duplication is performed on the minimizers to remove duplicate sequences; and repeated sequences may reduce the quality of the minimizers, so de-duplication is very important to evaluate the quality of the minimizer.
(b) Coverage: coverage is used as a scoring index, that is, the frequency and proportion of the minimizers in the substring with the length of k-mer; and the higher the coverage, the better the minimizer can represent the substring sequence with the length of k-mer, and the higher the quality.
(c) Similarity: similarity is used as a scoring index, that is, the similarity and coverage between the minimizers; and the higher the similarity, the stronger the representativeness between the minimizers, and the higher the quality.
(d) Length: the length of the minimizer is used as a scoring index, that is, the shorter the length of the minimizer, the more they can represent the commonness and similarity of the substring sequence with the length of k-mer, and the higher the quality.

It should be understood that the initially obtained minimizers are not optimal, and it takes many optimizations to obtain the optimal minimizers.
(3.3) The acquired minimizers are stored in a minimizer cache region and counted.

In this embodiment, the obtained minimizers are stored in the minimizer cache region, and at the same time, it is necessary to count the minimizers, which is helpful to determine the frequency and proportion of the minimizers in the substring sequence with the length of k-mer.

In this embodiment, the counting process is usually divided into two stages:

The first stage is the prediction stage: using the hash function and hash table, the minimizers are re-counted; in an embodiment, the substring sequence with the length of k-mer is mapped into the hash table, and the number of times that each minimizer appears in the substring sequence with the length of k-mer is counted; and the purpose of the pre-counting stage is to quickly calculate the frequency and proportion of appearance of the minimizers for subsequent counting and sorting.

The second stage is an accurate counting stage: after the pre-counting stage is completed, the minimizers are accurately counted by using optimization methods, such as a bitmap and a compressed hash table. In an embodiment, by using the bitmap or compressed hash table, the hash value and counting information of the minimizers are compressed into the bitmap or the hash table for quick query and update. In addition, technologies such as multithreading and distributed computing can be used to speed up the counting process.
(3.4) The read length traverser filters the minimizers smaller than the first preset threshold and larger than the second preset threshold after traversing all the characters in the read length, to obtain the filtered minimizers.

In this embodiment, the first preset threshold and the second preset threshold are user-defined, and the user-defined threshold refers to the threshold that the user can set by himself, that is, the minimum and maximum times that the minimizers appear in the substring sequence with the length of k-mer.

It should be understood that user-defined thresholds can be determined according to specific research purposes and data characteristics. In practical application, users usually need to choose the appropriate minimizer threshold according to the size, complexity, noise and other factors of the substring sequence with the length of k-mer. If the minimum threshold is set too small, it may produce a lot of noise and errors, resulting in inaccurate comparison and analysis results. If the minimum threshold is set too large, some important information may be ignored, thus affecting the results of comparison and analysis. Therefore, it is necessary to choose the appropriate minimizer threshold according to the specific situation.
(3.5) The seed finder (SF) reads the filtered minimizers output by the read length traverser to obtain the unfiltered minimizers.
(3.6) The seed finder queries the seed position of the unfiltered minimizers and the reference sequence information thereof from the index table.

In this embodiment, according to the hash value of the unfiltered minimizers, the seed finder queries the seed positions of the unfiltered minimizers in the substring sequence with the length of k-mer from the index table, and obtains the corresponding reference sequence information. It should be understood that the index table is the data structure for storing the seed position of the substring sequence with the length of k-mer and the corresponding reference sequence information, so that the seed position can be easily queried from the index table.
(3.7) The seed finder generates the reference subgraph according to the seed position of the unfiltered minimizes and the reference sequence information of the seed position of the unfiltered minimizers acquired in sub-step (3.6), and identifies candidate mapping positions according to the reference subgraph to filter the candidate mapping area.

In an embodiment, firstly, starting from the seed position, it expands to the left and right sides to obtain a sequence fragment with a certain length; then, according to the reference sequence information, the sequence fragment is mapped to the reference genome and converted into the reference subgraph. The reference subgraph is the directed acyclic graph, which consists of a series of nodes and edges, where each node represents the sequence and each edge represents the connection between adjacent sequences.

It should be understood that the generation process of the reference subgraph needs to consider many factors, such as sequence quality, sequence similarity, sequence length and so on. After that, a series of optimization operations will be carried out to improve the accuracy and efficiency of comparison and analysis. In an embodiment, the reference subgraphs are duplicated, merged and pruned to reduce noise and errors and improve the efficiency of comparison and analysis.
(4) Alignment: a PUM mode is adopted to run approximate string matching between the read length and all unfiltered candidate mapping positions, so as to complete the optimal alignment of the reference gene sequence and the query gene sequence.

In this embodiment, PIM is divided into two types, namely, processing-near-memory (PNM) and processing-using-memory (PUM). PNM adds PIM logic near or inside the memory. Generally, PIM logic is located in the logic layer or memory controller, in which the computing unit is integrated near or inside the memory. The programmable computing unit is placed in the memory bank, showing attractive low-latency and high-bandwidth memory access. PUM makes use of the inherent properties and operating principles of memory cells and cell arrays, and can compute through the interaction between cells. It processes the data in memory subarrays without reading the data.

It should be understood that during optimization, the distance threshold can also be set, and this distance-aware alignment can help to reduce intermediate data and speed up the analysis of the genome graph.
(4.1) The alignment instruction is flowed into the instruction cache region of a register clock driver (RCD) by adopting the PUM mode.

In this embodiment, the alignment instruction includes the bitwise comparison operation corresponding to the Bitmap method. The register clock driver (RCD) in the cache chip of DIMM is modified to support bitwise alignment. Command/address (C/A) signals issued by RCD can drive subarrays that perform batch bitwise operations.
(4.2) The alignment instruction is decoded by adopting the PUM instruction decoder added in RCD to acquire the subarray corresponding to the alignment instruction, and the data is loaded into the subarray.

In this embodiment, in order to support PIM, the ISA instruction is expanded, in which pnm_load and pnm_comp drive PNM commands, which are fed to SPU to load and query the index, thus completing the seed operation; pum_and, pum_or and pum_shift are PUM instructions, which are provided to RCD to generate DDR C/A single signals to perform bitwise operations. Other standard DDR instructions are issued by the memory controller to realize the normal memory access function. This embodiment allocates physically continuous memory blocks to the data to be processed in the memory, similar to the recent PIM work. The continuous distribution makes the address of the PIM instruction translated only once, and the rest addresses can be obtained by offset.
(4.3) Four pattern bit masks of A, G, T and C are generated for querying the read length based on the Bitmap method.

In this embodiment, the Bitmap method is a kind of ASM (approximate string matching) algorithm, which uses fast and simple bitwise operation, so it is easy to realize efficient hardware acceleration. The Bitmap method can calculate whether a given string contains the substring that is “about equal to” the given pattern string, where “about equal to” is defined by the Levinstein distance—if the distance between the substring and the pattern string is less than or equal to the given k, the algorithm considers them to be matching. Bitmap only uses fast and simple bitwise operation to execute ASM, which solves the problem of calculating the minimum editing distance between the reference genome with the maximum k errors and the read length.

In an embodiment, the first step is preprocessing, which firstly generates the pattern bit mask for each character of the pattern string; and these pattern bit masks help to represent the query mode in binary format. Then the editing distance is calculated. By using the preprocessed pattern bit mask, a state bit vector of the partial matching information of the text characters checked so far is updated and saved at each text iteration, and each text character is checked one by one by the bitwise operation.
(4.4) Each vertex of the reference subgraph is iterated, and the insertion, deletion, replacement and matching bit vectors of each vertex are calculated through the bitwise operation, so as to achieve the optimal alignment of the reference gene sequence and the query gene sequence.

In an embodiment, each vertex of the reference subgraph is iterated, and the sub-array is divided into three groups: a data group, a control group and a bitwise group. The data group corresponds to the row used for storing conventional data, the control group contains four pattern bit mask vectors and two pre-initialized rows to control a bitwise alignment operation, and the bitwise group consists of rows that perform the bitwise operation in the row parallel manner. The insertion, deletion, replacement and matching bit vectors of each vertex are calculated by the data group, the control group and the bitwise group and the bitwise operation, so as to achieve the optimal alignment of the reference gene sequence and the query gene sequence.

In this embodiment, the bitwise operations include AND, OR and SHIFT, etc.
(4.4.1) Computing of the insertion bit vector: when the character in the query sequence is inserted into the reference sequence, the insertion bit vector needs to be calculated. In an embodiment, firstly, the bit vectors before and after the insertion position in the query sequence are subjected to an OR operation to determine whether the bit vectors before and after the insertion position are the same; then, the bit vector behind the insertion position will be moved back one bit to make room for the insertion bit vector; and finally, the value of the insertion bit vector is the bit vector corresponding to the inserted characters in the query sequence.
(4.4.2) Computing of the deletion bit vector: when the character in the reference sequence is deleted, the deletion bit vector needs to be calculated. In an embodiment, firstly, the bit vectors before and after the deletion position in the reference sequence are subjected to the OR operation to determine whether the bit vectors before and after the deletion position are the same; then the bit vector behind the deletion position moves forward by one bit to fill the vacancy left by the deletion position; finally, a bit vector whose value is all 0 is deleted.
(4.4.3) Computing of the replacement bit vector: when the character in the reference sequence is replaced, the replacement bit vector needs to be calculated. In an embodiment, firstly, the bit vectors before and after the replacement position in the reference sequence and the bit vectors before and after the replacement position in the query sequence are subjected to the OR operation to determine whether the bit vectors before and after the replacement position are the same; then the bit vector behind the replacement position in the query sequence is moved backward by one bit to make room for the replacement bit vector; finally, the value of the replacement bit vector is the bit vector corresponding to the replaced character in the query sequence.
(4.4.4) Computing the matching bit vector: when the character in the reference sequence and the query sequence matches, the matching bit vector needs to be calculated. In an embodiment, firstly, the bit vectors at corresponding positions in the reference sequence and the query sequence are subjected to the AND operation to determine whether their values are the same; if they are the same, the bit vector whose value is all 1 is matched; if they are different, the bit vector whose value is all 0 is matched.

Through the above computing, the comparison result between the reference sequence and the query sequence can be obtained. In an embodiment, by comparing the bit vectors in the same position in the reference sequence and the query sequence, if their values are the same, it means that the characters in this position match; and if their values are different, it means that the characters in this position need to be inserted, deleted or replaced. In this way, the reference sequence and the query sequence are compared, and the comparison results are output, so that the best comparison information can be obtained and returned to the host.

The present disclosure innovatively integrates processing-near-memory and in-situ computing, and carries out low-cost modification; according to the present disclosure, a customized seed processing unit is placed beside each DRAM level to explore the parallelism of the levels, so that the low access delay can reduce the irregular memory access overhead in the seeding step; the present disclosure takes the index as the center, which effectively reduces the transmission of index data between rows and columns; according to the present disclosure, the row access command sequence of the modified subarray structure is specialized, and the row parallel in-situ computing ability is allowed to adjust the bitwise operation in the alignment step; the present disclosure further introduces the distance sensing technology to eliminate the complex data dependence in the genome graph; the present disclosure expands the instruction set to support customized memory operation; and the present disclosure can accelerate the analysis of the genome graph.

Corresponding to the aforementioned embodiment of the genome graph analysis method based on in-memory computing, the present disclosure also provides an embodiment of a genome graph analysis device based on in-memory computing.

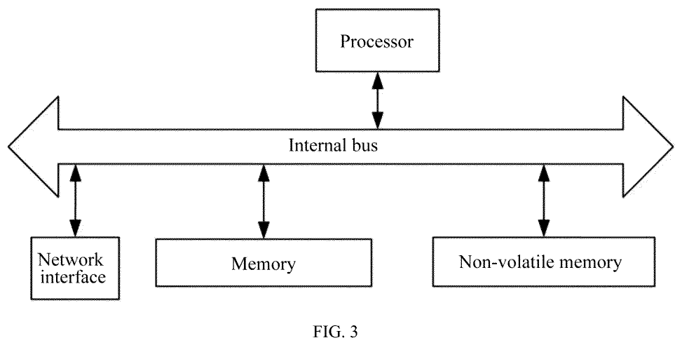

Referring to FIG. 3, a genome graph analysis device based on in-memory computing provided by an embodiment of the present disclosure includes one or more processors for implementing the genome graph analysis method based on in-memory computing in the above embodiment.

The embodiment of the genome graph analysis device based on in-memory computing of the present disclosure can be applied to any equipment with data processing capability, which can be equipment or devices such as computers. The embodiment of the device can be realized by software, or by hardware or a combination of hardware and software. Taking software implementation as an example, as a logical device, it is formed by reading the corresponding computer program instructions in the non-volatile memory into the memory through the processor of any equipment with data processing capability. From the hardware level, as shown in FIG. 3, it is a hardware structure diagram of any equipment with data processing capability where the genome graph analysis device based on in-memory computing of the present disclosure is located. Besides the processor, memory, network interface and nonvolatile memory shown in FIG. 3, any equipment with data processing capability where the device is located in the embodiment usually includes other hardware according to the actual functions of the equipment with data processing capability, which will not be described here again

The implementing process of the functions and functions of each unit in the above-mentioned device is detailed in the realization process of the corresponding steps in the above-mentioned method, and will not be repeated here.

For the device embodiment, because it basically corresponds to the method embodiment, it is only necessary to refer to the partial description of the method embodiment for the relevant points. The device embodiment described above is only schematic, in which the units described as separate components may or may not be physically separated, and the components displayed as units may or may not be physical units, that is, they may be located in one place or distributed to multiple network units. Some or all of the modules can be selected according to actual needs to achieve the purpose of the solution of the present disclosure. Those skilled in the art can understand and implement the present disclosure without creative labor.

The embodiment of the present disclosure also provides a computer-readable storage medium, on which a program is stored, and when executed by a processor, the program implements the genome graph analysis method based on in-memory computing in the above embodiment.

The computer-readable storage medium can be an internal storage unit of any equipment with data processing capability as described in any of the previous embodiments, such as a hard disk or a memory. The computer-readable storage medium can also be any equipment with data processing capability, such as a plug-in hard disk, a Smart Media Card (SMC), an SD card, a Flash Card and the like. Further, the computer-readable storage medium can also include both internal storage units and external storage devices of any device with data processing capability. The computer-readable storage medium is used for storing the computer program and other programs and data required by any equipment with data processing capability, and can also be used for temporarily storing data that has been output or will be output.

The above embodiments are only used to illustrate the design ideas and characteristics of the present disclosure, and their purpose is to enable those skilled in the art to understand the contents of the present disclosure and implement it accordingly. The protection scope of the present disclosure is not limited to the above embodiments. Therefore, all equivalent changes or modifications made according to the principles and design ideas disclosed in the present disclosure are within the protection scope of the present disclosure.

What is claimed is:

1. A genome graph analysis method based on in-memory computing, comprising:
step (1) construction: combining a linear reference genome with genetic variation to construct a genome graph;
step (2) indexing: generating indexes for a plurality of vertices of the genome graph, and constructing an index table according to the generated indexes;
step (3) seeding: dividing a read length into a plurality of substrings with a length of k-mer, querying the index table to obtain a seed position, generating a reference subgraph according to the seed position, and identifying a candidate mapping position according to the reference subgraph to filter a candidate mapping area;
sub-step (3.1) receiving, by the read length traverser, an input read length from a host, wherein a seed processing unit comprises a read length traverser and a seed finder;
sub-step (3.2) traversing, by the read length traverser, the input read length in order to obtain a plurality of minimizers, and scoring the plurality of minimizers according to a scoring mechanism to obtain a plurality of optimized minimizers;
sub-step (3.3) storing the acquired minimizers in a minimizer cache region and counting the minimizers;
sub-step (3.4) filtering, by the read length traverser, minimizers smaller than a first preset threshold and minimizers larger than a second preset threshold after traversing all characters in the read length, to acquire filtered minimizers;
sub-step (3.5) reading, by the seed finder, the filtered minimizers output by the read length traverser to obtain unfiltered minimizers;
sub-step (3.6) querying, by the seed finder, a seed position of the unfiltered minimizers and a reference sequence information of the seed position of the unfiltered minimizers from the index table; and
sub-step (3.7) generating, by the seed finder, the reference subgraph according to the seed position of the unfiltered minimizes and the reference sequence information of the seed position of the unfiltered minimizers acquired in the sub-step (3.6), and identifying the candidate mapping position according to the reference subgraph to filter the candidate mapping area; and
step (4) alignment: running approximate string matching between the read length and all unfiltered candidate mapping positions by adopting a Process-Using-Memory (PUM) mode, so as to achieve an optimal alignment of a reference gene sequence and a query gene sequence.

2. The genome graph analysis method based on in-memory computing according to claim 1, wherein the step (1) further comprises:
sub-step (1.1) acquiring a basic information of the linear reference genome using a genome browser software, wherein the basic information comprises a genome sequence and a gene annotation information;
sub-step (1.2) sequencing and analyzing the basic information to acquire a genetic variation information;
sub-step (1.3) combining the genetic variation information with the genome sequence of the linear reference genome by adopting a comparison-based method and an assembly-based method to acquire individual genome sequences and a variation information in the individual genome sequences;
sub-step (1.4) annotating genome on the variation information in the individual genome sequences by a gene annotation tool to acquire an annotation information; and
sub-step (1.5) combining the linear reference genome, the individual genome sequences and the annotation information to construct a genome graph.

3. The genome graph analysis method based on in-memory computing according to claim 1, wherein the step (2) further comprises:
sub-step (2.1) calculating, for each genome graph, hash values of all vertices in the genome graph and mapping the hash values into one bucket;
sub-step (2.2) sorting, for each bucket, all the vertices in the bucket according to the hash values, and assigning one index to each vertex;
sub-step (2.3) adding, for each vertex, an index of each vertex in the each genome graph to a corresponding hash table entry; and
sub-step (2.4) sorting all hash table entries according to vertex identifiers and storing the hash table entries in the constructed index table.

4. (canceled)

5. The genome graph analysis method based on in-memory computing according to claim 1, wherein the minimizers are a set of sequences in substrings with the length of k-mer, and the minimizers satisfy the following conditions: each of the minimizers appears at least twice in the substrings with the length of k-mer; each of the minimizers are not capable of being a subsequence of other minimizers; and a sum of lengths of all the minimizers is less than or equal to a total length of the sequences in the substrings with the length of k-mer.

6. The genome graph analysis method based on in-memory computing according to claim 1, wherein the step (4) comprises:
sub-step (4.1) flowing an alignment instruction into an instruction cache area of a register clock driver by adopting the PUM mode;
sub-step (4.2) decoding the alignment instruction by adopting a PUM instruction decoder added in the register clock driver to acquire a subarray corresponding to the alignment instruction, and loading data into the subarray;
sub-step (4.3) generating four pattern bit masks of A, G, T and C for querying the read length based on a bitmap method; and
sub-step (4.4) iterating each vertex of the reference subgraph, and calculating insertion, deletion, replacement and matching bit vectors of the each vertex by a bitwise operation, so as to achieve the optimal alignment of the reference gene sequence and the query gene sequence.

7. The genome graph analysis method based on in-memory computing according to claim 6, wherein the sub-step (4.3) further comprises: preprocessing, comprising generating one pattern bit mask for each character of a pattern string; and calculating an editing distance, updating and saving a state bit vector of a partial matching information of text characters checked so far at each text iteration using the preprocessed pattern bit mask, and checking each of the text characters one by one by the bitwise operation.

8. The genome graph analysis method based on in-memory computing according to claim 6, wherein the sub-step (4.4) further comprises:
iterating the each vertex of the reference subgraph, and dividing the subarray into three groups: a data group, a control group and a bitwise group, wherein the data group corresponds to a row for storing conventional data, the control group comprises four pattern bit mask vectors and two pre-initialized rows to control a bitwise alignment operation, and the bitwise group comprises rows that perform the bitwise operation in a row parallel manner; and
calculating the insertion, the deletion, the replacement and the matching bit vectors of the each vertex by the data group, the control group and the bitwise group as well as the bitwise operation, to achieve the optimal alignment of the reference gene sequence and the query gene sequence.

9. A genome graph analysis device based on in-memory computing, comprising one or more processors for implementing the genome graph analysis method based on in-memory computing according to claim 1.

10. A non-transitory computer-readable storage medium on which a program is stored, wherein the program, when executed by a processor, is configured to implement the genome graph analysis method based on in-memory computing according to claim 1.
